(19) United States
(12) Patent Application Publication
(10) Pub. No.: US 2006/0293631 A1
(43) Pub. Date: Dec. 28, 2006

(54) CATHETER HOLDER

(76) Inventor: Sherry L. Bolt, Longview, TX (US)

Correspondence Address: LITMAN LAW OFFICES, LTD, PO BOX 15035, CRYSTAL CITY STATION, ARLINGTON, VA, 22215, US

(21) Appl. No.: 11/167,299

(22) Filed: Jun. 28, 2005

(51) Int. Cl. A61F 5/44

(52) U.S. Cl. 604/353; 604/322

(57) ABSTRACT

A catheter urine collection bag with a wide elastic sleeve worn on the upper thigh of the patient. The sleeve is connected by fabric straps to a belt worn around the patient's waist. The fabric straps may be part of a garter belt assembly. A fabric pouch holds the urine collection bag. A portion of the pouch body includes elastic fabric serving holding the bag firmly when filling. The top of the pouch is open to accept the collection bag. Two Velcro tabs on the top of the pouch are positioned to be fastened over the shoulders of the bag to hold it in the pouch. Holes at the bottom of the holder accept the nozzle and valve on the bag that empties the bag as necessary, while a nozzle in the top of the bag is connected to the catheterization tubing of the patient.

### BACKGROUND OF THE INVENTION

1. Field of the Invention

The present invention relates to support means for holding the urine collection bag of catheterized patients. The invention particularly relates to elastic leg wraps for supporting urine collection bags.

2. Description of the Related Art

Patients afflicted with urological disorders frequently must resort to the continuous passing of urine by catheterization of the urethra. The requirement for catheterization may last for short periods of times measured in days or for very much longer periods of time. In either event, the passed urine is collected continuously through a plastic catheterization tubing into a plastic bag equipped with an inlet only check valve and an outlet plastic on-off nozzle for emptying the bag.

Catheterization presents many problems for the patient with the absolute need to prevent infections originating from the catheterization process being a paramount requirement. But the mechanics of living with a urine collection bag presents problems which are quite serious in and of themselves. When nearly full the bag is bulky and heavy. It creates a significant downward force on the straps used in prior art urine collection bags for holding. the bag on the patients leg. The bag can begin to slip downward and pull on the catheter tubing This can lead to dislodgement of the catheter itself requiring the remedial attention of a physician and more discomfort for the patient.

A bulky collection bag presents its own hazard, especially for ambulatory patients. It offers more opportunities for striking an obstruction with urine spillage or dislodgement a real possibility. The simple act of walking with a collection bag attached to the persons leg can magnify the forces that tend to dislodge the collection bag. When walking the urine collection bag can make an embarrassing sloshing sound which prior art collection bags do not dampen. Once again, these problems are not overcome by prior art catheter systems and collection bag holders.

When urine collection bags must be worn for extended periods and are not firmly secured to the patient it is known that skin irritation and chafing associated with movement of the collection bag on the patients leg can occur. This just adds more discomfort to an already discomfortable situation. Certainly, that discomfort is worsened for the patient by the constant anxiety over the security of the means used in the prior art to reliably support the collection bag and avoid a major failure accompanied by a large urine spill and the possible loss of catheter retention in the urethra.

None of the inventions and patents of the prior art, taken either singly or in combination, is seen to describe the instant invention as claimed herein. Thus, a catheter bag holder solving the aforementioned problems is desired.

### SUMMARY OF THE INVENTION

The support structure of the invention for the urine collection bag is comprised of several elements. The first is an elastic sleeve prepared to fit securely high up on the thigh of the catheterized patient and extending down the thigh to end just above the knee. The broad area of the elastic sleeve establishes a stable base of support for a urine collection bag holder to be attached. But in addition, the top of the sleeve is removably connected by straps or belts to a closed belt worn around the waist of the patient. The combination of elastic sleeve and straps connected to a belt eliminates any problem of slippage downward that may be experienced by a urine collection bag holder.

The urine collection bag holder is attached as a pocket well toward the top of the sleeve and close to the interior side of the thigh. A window of stretchable fabric is included in the exterior fabric wall of the pocket to accommodate any expansion of the collection bag as it fills. The top of the pocket is open to receive a plastic collection bag. The bottom of the pocket has at least one hole through which a nozzle and valve means is inserted. Optionally, there may be two apertures or button holes at the bottom of the holder. The nozzle and valve is connected to the bottom of the bag to empty the bag of urine. The top of the bag contains a nozzle for connection with the catheter tubing. The nozzle itself may contain a check valve to prevent any backflow of urine from the collection bottle into the catheter tubing and urethra. Connected to the top of the pocket are two Velcro tabs. These Velcro tabs pass over the shoulders of the collection bag in the pocket and are attached firmly to the elastic sleeve. The Velcro tabs prevent the collection bag from falling out of the pocket.

These and other features of the present invention will become readily apparent upon further review of the following specification and drawings.

### BRIEF DESCRIPTION OF THE DRAWINGS

FIG. 1 is an environmental perspective view of a catheter urine collection bag holder displayed as installed according to the present invention.

FIG. 2 is a side view of the elastic sleeve, belt and straps as installed to support the catheter bag holder of the invention.

FIG. 3A is a frontal view of the catheter urine collection bag of the invention containing a regular collection bag.

FIG. 3B is a frontal view of the catheter urine collection bag holder of the invention containing an optional collection bag.

FIG. 4 is a frontal display of the collection bag holder on the elastic sleeve and two different types of commercial collections bags for use with the collection bag holder of the invention.

Similar reference characters denote corresponding features consistently throughout the attached drawings.

### DETAILED DESCRIPTION OF THE PREFERRED EMBODIMENTS

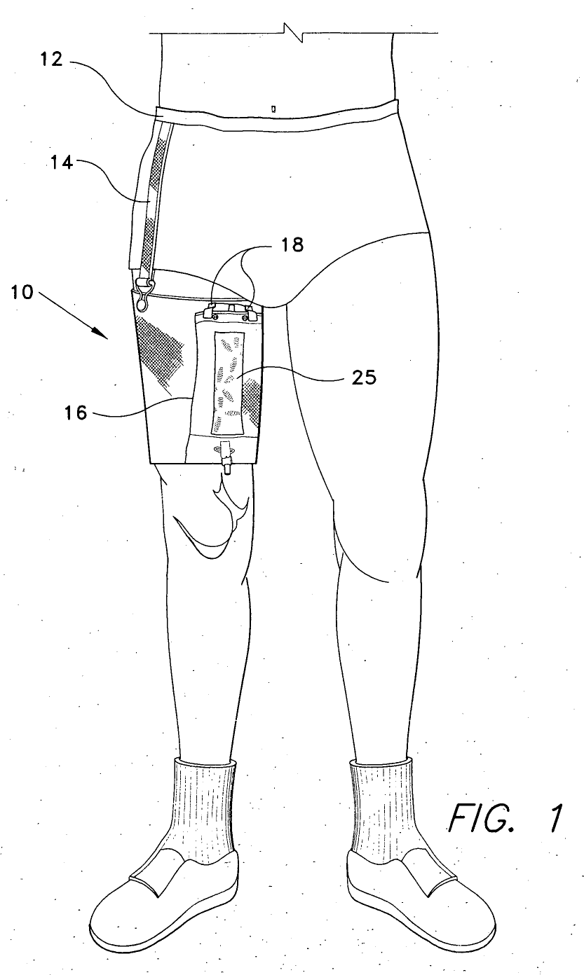

Referring to FIG. 1, the elastic sleeve (10) of the invention is shown in position high up on the patients thigh. The sleeve is coupled to a belt (12) about the waist of a patient with one or more garters (14) closed on and attached to the upper edge of the sleeve itself. The sleeve covers most of the thigh and completes the mechanism by which the urine bag holder and the collection bag are supported. The sleeves elasticity is augmented by the garter straps which provide strong and comfortable support for even full collection bags. Any propensity for the bag support to slip down or for the sleeve to rollup is overcome. As FIG. 1 shows, the bag holder (16) is attached to the elastic sleeve (10) near the top and bottom edges of the holder, preferably by sewing the holder to the sleeve around the perimeter of the bag holder. However, other means such as snap on fasteners or Velcro tabs are available to fasten the two parts together, particularly if it is desirable to replace or remove the bag holder for repair or washing.

Preferably, two Velcro tabs are attached to the top of the bag holder (18). When the bag is inserted in the holder, these two tabs are pressed onto the sleeve over the shoulders of the collection bag to secure the bag in the bag holder. Another feature of the invention is the incorporation of elastic fabric (25) as a section of the covering of the bag holder. This allows the volume of the collection bag holder to increase consistent with the increase in the volume of the collection bag at it fills. The elastic section of the bag cover holds a near filled collection bag more firmly in the bag holder. The elastic section is inserted in the bag holder by sewing or with adhesives or other fasteners known in the art.

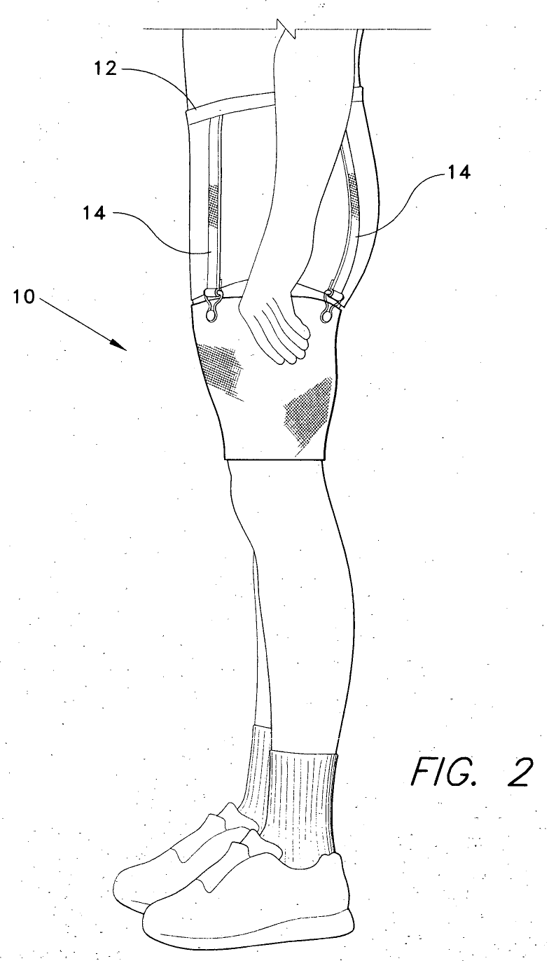

Referring to FIG. 2, a patient side view is presented on the installation of the elastic sleeve and the garter belt straps. The garter belt itself preferably includes Velcro tabs to close and hold the belt around the patient's waist. Material for producing the elastic sleeve (10) is readily available. Elastic fabrics are well known in the art. They have been produced for many years as copolymers of polyesters and urethanes and other comonomers. Major manufactures produce a variety of types with a variety of elastic strength not only for clothing but for medical uses as well.

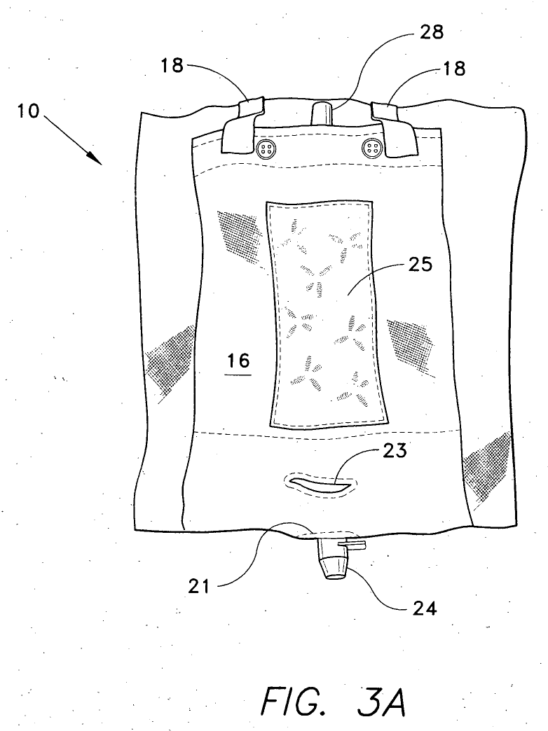

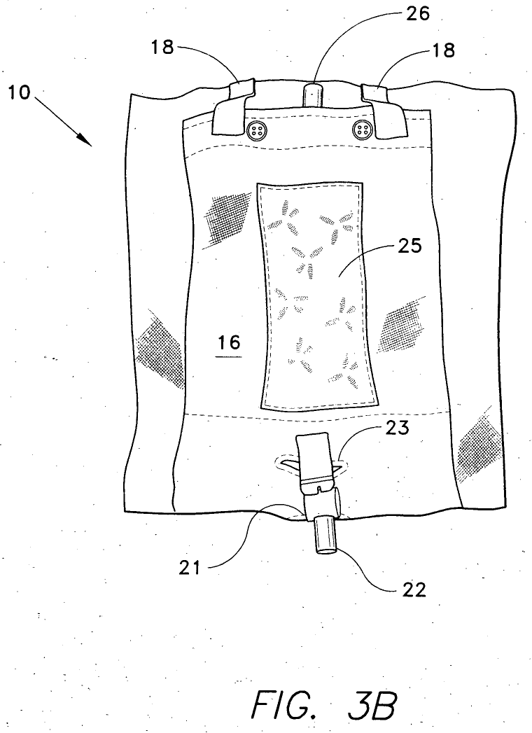

Referring to FIGS. 3A and 3B, the urine collection bag holder (16) of the invention is shown attached to the elastic sleeve (10), including the elastic section (25) of the holder. However, the holders are carrying two different types of typical collection bags evidenced by the two different types of nozzles (22) (24) inserted through apertures in the bottom of the holders. Alternative apertures in the bottom of the holder are shown as (23), providing two discharge apertures per bag. These nozzles are used to empty the urine collection bags. The bags are connected to the catheter tubing at valves (26) and (28). These valves are one way valves designed to prevent backup flow of urine into the catheter tubing.

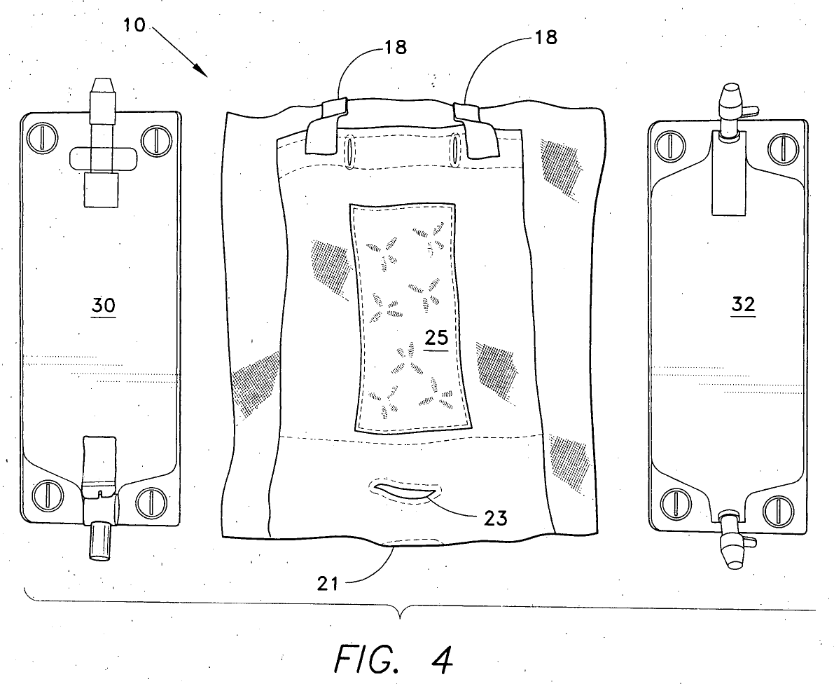

Referring to FIG. 4, a display is presented of two different types of urine collection bags (28) and (30) and the urine collection bag holder (25) secured on the elastic sleeve (10) of the invention. The primary difference in the collection bags is the type of inlet valves and outlet valves they use. These collection bags are well known in the art and are suitable for use with the collection bag holder of the invention. Both bags will be secured in the holder by the Velcro tabs and the elastic fabric patch (25). Both shall be emptied through one of two apertures (23) (24) in the bottom of catheter urine collection bag holder.

What is described herein is a urine collection bag holder for catheterized patients comprising a tubular thigh sleeve comprising an elastic fabric. The sleeve further includes an elongated fabric pocket attached to the sleeve with the pocket having an opening for receiving and containing collection bag and bottom apertures for emptying the collection bag. A means is included to attach and secure the top of the pocket to the collection bag.

It is to be understood that the present invention is not limited to the embodiments described above, but encompasses any and all embodiments within the scope of the following claims.

1-5. (canceled)

6. A urine collection bag holder for catheterized patients, comprising:
a tubular sleeve made of an elastic material, said sleeve adapted for being placed around a patient's thigh;
an elongated fabric pocket attached to the sleeve, said pocket having a top opening for receiving and containing a collection bag and at least one bottom aperture for emptying the collection bag;
means attached to the pocket for securing the top of the pocket over the collection bag; and
means for preventing slippage of the sleeve, wherein said means for preventing slippage includes garters connected to the sleeve and further connected to a garter belt adapted to he worn around a patient's waist.

7. (canceled)

8. The collection bag holder of claim 6 wherein said fabric pocket is selected from the group consisting of cotton, polyester, wool, polyamides and polyethers.

9. The collection bag holder of claim 6 having two bottom apertures for emptying the collection bag.
